(19) United States
(12) Patent Application Publication
(10) Pub. No.: US 2026/0053368 A1
(43) Pub. Date: Feb. 26, 2026

(54) PHYSIOLOGICAL SIGNAL MEASURING DEVICE AND METHOD

(71) Applicants: BRIGHTON PRECISION ENGINEERING CO., LTD., Taoyuan City (TW); ZEBRIGHT INTELLIGENT TECH CO., LTD., Taoyuan City (TW)

(72) Inventor: FENG JU CHUANG, Hsinchu City (TW)

(21) Appl. No.: 19/225,061

(22) Filed: Jun. 2, 2025

(30) Foreign Application Priority Data
Aug. 23, 2024 (TW) 113131768

(51) Int. Cl. A61B 5/00

(52) U.S. Cl. CPC A61B 5/0075; A61B 5/7235

(57) ABSTRACT

A physiological signal measuring device is provided. The physiological signal measuring device includes a light-emitting module, a light sensing module, and a controller. The light-emitting module includes a light-emitting unit configured to generate emitted light according to a first control signal in each measurement period. The light sensing module includes a sensing unit configured to receive reflected light corresponding to the emitted light in each of the measurement periods. The controller is configured to change the first control signal in each of the measurement periods to change a wavelength of the emitted light, and obtain an optical parameter analysis result corresponding to the wavelength of the emitted light according to the reflected light. The controller is configured to obtain a physiological signal corresponding to at least one physiological parameter according to a spectral distribution curve composed of the optical parameter analysis results of the measurement periods.

### CROSS-REFERENCE TO RELATED APPLICATION

This application claims priority of Taiwan application No. 113131768 filed on Aug. 23, 2024, which is incorporated by reference in its entirety.

### TECHNICAL FIELD

The present disclosure relates to a physiological signal measuring device and method, and in particular, to a non-intrusive biophysiological signal measuring device and method.

### BACKGROUND

With the attention to health, people's demand for monitoring various physiological signals of the body at any time is increasing. For a plurality of types of physiological signals, for example, heart rate, blood oxygen, blood glucose, and blood pressure, a non-intrusive reflective optical measuring device may be put close to or in contact with a skin surface, emit light to illuminate the skin of a user and reach the tissue inside the skin, and receive reflected light reflected by the skin, to obtain a physiological signal of the user.

Therefore, it is important to improve the accuracy of the physiological signal measuring device and method.

### BRIEF SUMMARY OF THE DISCLOSURE

The present disclosure provides a physiological signal measuring device. The physiological signal measuring device includes a light-emitting module, a light sensing module, and a controller. The light-emitting module includes a light-emitting unit configured to generate emitted light according to a first control signal in each measurement period. The light sensing module includes a sensing unit configured to receive reflected light corresponding to the emitted light in each of the measurement periods. The controller is configured to change the first control signal in each of the measurement periods to change a wavelength of the emitted light, and obtain an optical parameter analysis result corresponding to the wavelength of the emitted light according to the reflected light. The controller is configured to obtain a physiological signal corresponding to at least one physiological parameter according to a spectral distribution curve composed of the optical parameter analysis results of the measurement periods.

Furthermore, the present disclosure provides a physiological signal measuring method. The physiological signal measuring method includes: arranging a light-emitting diode and a photodiode at a fixed distance; providing a measurement condition; driving the light-emitting diode in each measurement period according to the measurement condition, to generate emitted light; receiving, through the photodiode, reflected light corresponding to the emitted light in each of the measurement periods according to the measurement condition, and obtaining an optical parameter analysis result corresponding to the wavelength of the emitted light according to the reflected light; and when the number of the optical parameter analysis results of the measurement periods is less than a specific value, adjusting the measurement condition in each of the measurement periods, so as to generate emitted light corresponding to the adjusted measurement condition in each of the measurement periods.

In conclusion, the physiological signal measuring device and method can change a measurement condition to obtain a spectral distribution curve composed of a plurality of optical parameter analysis results, so that an absorption parameter and a scattering parameter of a physiological tissue can be accurately analyzed.

### BRIEF DESCRIPTION OF THE DRAWINGS

Aspects of the present disclosure are best understood from the following detailed description when read with the accompanying figures. It should be noted that, in accordance with the standard practice in the industry, various features are not drawn to scale. In fact, the dimensions of the various features may be arbitrarily increased or reduced for clarity of discussion.

FIG. 1 is a schematic diagram of an operation principle of a physiological signal measuring device according to an embodiment of the present disclosure.

FIG. 2 is a schematic circuit diagram of a physiological signal measuring device according to an embodiment of the present disclosure.

FIG. 3 shows spectral distribution curves of absorption parameters of glucose aqueous solutions with different glucose concentrations according to an embodiment of the present disclosure.

FIG. 4 is a schematic waveform diagram of a signal of a physiological signal measuring device according to an embodiment of the present disclosure.

FIG. 5 is a schematic circuit diagram of a physiological signal measuring device according to an embodiment of the present disclosure.

FIG. 6 is a flowchart of a physiological signal measuring method according to an embodiment of the present disclosure.

### DETAILED DESCRIPTION

The following disclosure provides many different specific embodiments for implementing different features of the provided patentable subject matter. Specific examples of components and configurations described below are used to simplify the present invention. Certainly, these examples are merely examples rather than limitations. In addition, in the present disclosure, reference numerals and/or symbols may be repeated in various embodiments. This repetition is for brevity and clearer reasons, and does not require a relationship between the various specific embodiments and/or configurations to be discussed.

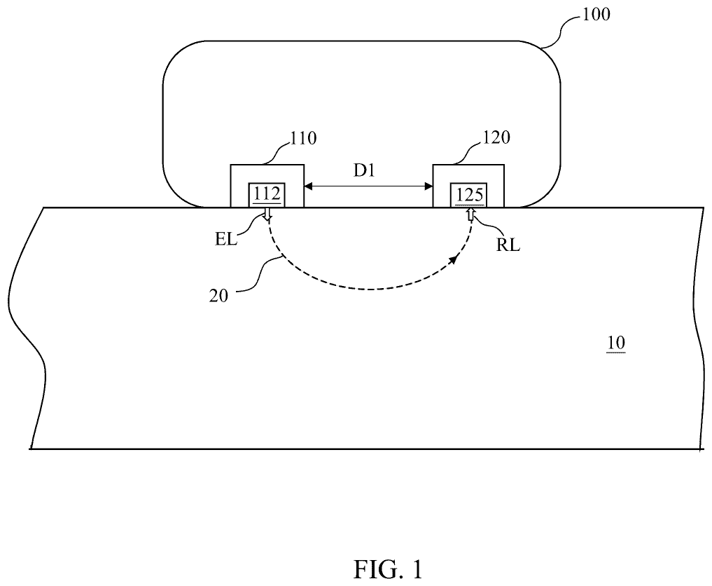

FIG. 1 is a schematic diagram of an operation principle of a physiological signal measuring device 100 according to an embodiment of the present disclosure. The physiological signal measuring device 100 is placed on skin of a body (for example, an arm) of a user 10 for measurement. The physiological signal measuring device 100 includes a light-emitting module 110 and a light sensing module 120. A light-emitting unit 112 of the light-emitting module 110 provides emitted light EL to the skin of the user 10. The emitted light EL penetrates the skin to cells, blood vessels, and the like in the body of the user 10, and is partially absorbed, partially scattered, and partially reflected by the cells and blood vessels in the body. The light sensing module 120 receives reflected light RL from the user. The physiological signal measuring device 100 measures and analyzes the reflected light RL received by a sensing unit 125 of the light sensing module 120, and then obtains a physiological signal of the user, for example, blood glucose, blood oxygen, blood pressure, heart rate, blood lipid, bilirubin, lactic acid, respiratory frequency, heart rate variability, core body temperature, body water, alcohol, urea, creatinine, albumin, hemoglobin, glycated hemoglobin, collagen, or neutrophil. As shown in FIG. 1, the light-emitting module 110 and the light sensing module 120 are arranged at a fixed distance D1 from each other. In other words, a distance between the light-emitting unit 112 and the sensing unit 125 is fixed. In some embodiments, the fixed distance D1 is equal to 5 millimeters. In some embodiments, the fixed distance D1 is less than 5 millimeters.

In some embodiments, the light-emitting unit 112 is a single light-emitting diode (LED), and the sensing unit 125 is a single photodiode (PD). In addition, there is a light propagation path 20 between the light-emitting unit 112 and the sensing unit 125. In response to different driving current, the emitted light EL generated by the light-emitting unit 112 has a different wavelength, that is, the wavelength (for example, a peak wavelength) of the emitted light EL drifts as the driving current changes. For example, when the driving current increases, the peak wavelength of the emitted light EL drifts toward a long wavelength, for example, the peak wavelength increases. Conversely, when the driving current decreases, the peak wavelength of the emitted light EL drifts toward a short wavelength, for example, the peak wavelength decreases.

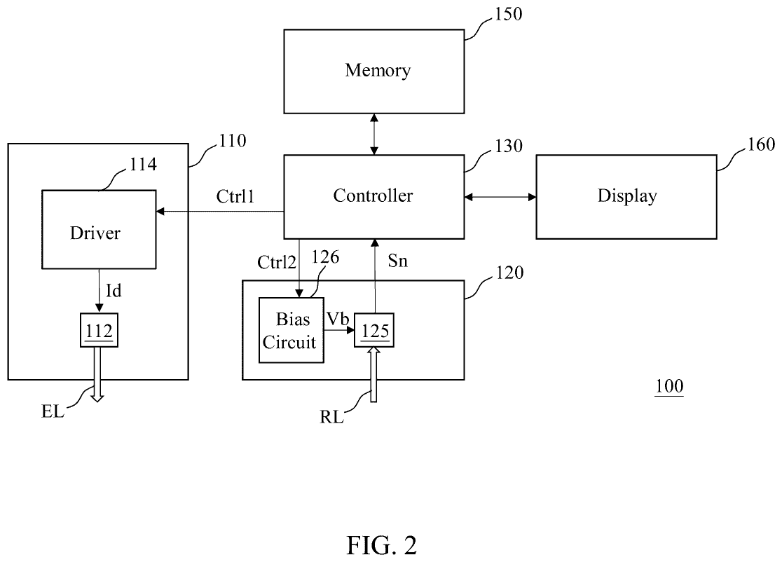

FIG. 2 is a schematic circuit diagram of a physiological signal measuring device 100 according to an embodiment of the present disclosure. The physiological signal measuring device 100 includes a light-emitting module 110, a light sensing module 120, a controller 130, a memory 150, and a display 160. The light-emitting module 110 includes a light-emitting unit 112 and a driver 114. The light sensing module 120 includes a sensing unit 125 and a bias circuit 126. In some embodiments, the light-emitting unit 112 is a light-emitting diode (for example, a monochrome light-emitting diode), and the sensing unit 125 is a photodiode.

The controller 130 provides a control signal Ctrl1 to the driver 114, to control the driver 114 to provide a driving current Id to the light-emitting unit 112, to generate emitted light EL and control duration of the emitted light EL. In response to the emitted light EL, the sensing unit 125 receives corresponding reflected light RL, and converts photons of the reflected light RL into a sensing signal Sn. In addition, the controller 130 provides a control signal Ctrl2 to the bias circuit 126, to control the bias circuit 126 to provide a fixed bias voltage Vb to the sensing unit 125. After obtaining the sensing signal Sn, the controller 130 may analyze the sensing signal Sn to obtain an optical parameter analysis result, and establish a spectral distribution curve about an absorption parameter μₐ and/or a scattering parameter μₛ according to a plurality of optical parameter analysis results. As previously described, when the driving current Id changes, the wavelength of the emitted light EL drifts. Therefore, adjusting the driving current Id can cause the light-emitting unit 112 to generate emitted light EL of a different wavelength. Therefore, by changing the driving current Id, the controller 130 may establish a spectral distribution curve about an absorption parameter μₐ and a scattering parameter μₛ according to reflected light RL with a different wavelength. Through a spectral distribution curve composed of more wavelengths, the controller 130 may analyze an absorption parameter μₐ and a scattering parameter μₛ of a physiological tissue more accurately, to obtain a corresponding physiological signal of the user 10.

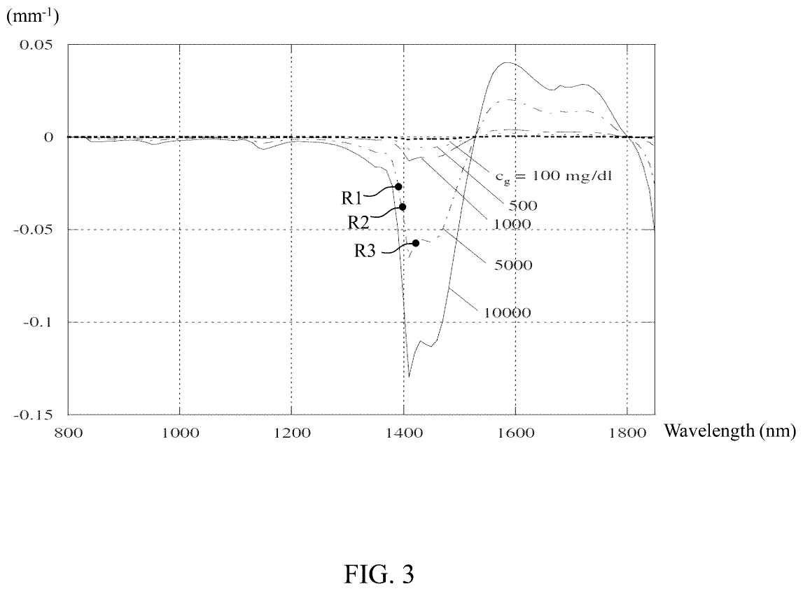

In some embodiments, the controller 130 may be a processor capable of executing an algorithm. According to a model stored in the memory 150 and settings and parameters thereof, the controller 130 may establish a corresponding spectral distribution curve according to the sensing signal Sn. For example, FIG. 3 shows spectral distribution curves of absorption parameters μₐ of glucose aqueous solutions with different glucose concentrations C_{g}. In some embodiments, the spectral distribution curves in FIG. 3 are results of a spectrum of an absorption parameter μₐ changing with a glucose concentration C_{g}, where the results are obtained by performing Monte Carlo simulation on light propagation in a wavelength range of 1200 nm to 1800 nm and are stored in the memory 150.

FIG. 3 shows spectral distribution curves of absorption parameters μₐ with glucose concentrations C_{g} of 100, 500, 1000, 5000 and 10000 mg/dl respectively. A glucose band increases around a wavelength of 1600 nm, and a water band decreases around a wavelength of 1400 nm. In some embodiments, As the wavelength of the emitted light EL drifts with the driving current Id, the controller 130 may obtain a spectral distribution curve of an absorption parameter ula of the user 10 according to the reflected light RL received by the sensing unit 125. By comparing the spectral distribution curve of the absorption parameter ula of the user 10 with the spectral distribution curve of the absorption parameter μₐ of the known glucose concentration C_{g} shown in FIG. 3, the controller 130 may obtain the absorption parameter μₐ of the glucose concentration C_{g} of the user 10.

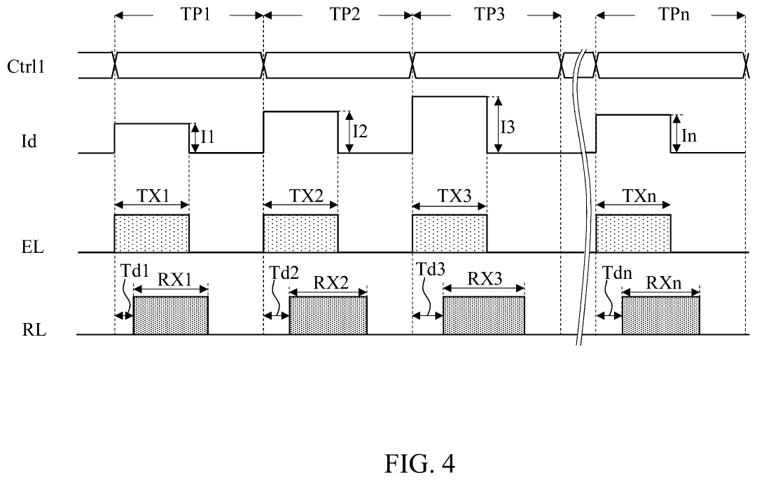

FIG. 4 is a schematic waveform diagram of a signal of a physiological signal measuring device 100 according to an embodiment of the present disclosure. Referring to FIG. 2 and FIG. 4 together, the controller 130 provides a control signal Ctrl1 to the driver 114 in each of measurement periods TP1 to TPn. The control signal Ctrl1 includes information about drive settings of the light-emitting unit 112, for example, a current value of the driving current Id and drive duration. In response to the control signal Ctrl1, the driver 114 provides the driving current Id to the light-emitting module 110, to control the light-emitting unit 112 to generate emitted light EL, and a wavelength of the emitted light EL corresponds to the current value of the driving current Id. In some embodiments, the measurement periods TP1 to TPn have a same time interval. In some embodiments, the measurement periods TP1 to TPn have variable time intervals. For example, the controller 130 may dynamically adjust each time interval of the measurement periods TP1 to TPn according to the optical parameter analysis result of the sensing signal Sn. In some embodiments, the time intervals of the measurement periods TP1 to TPn are less than or equal to one minute. In addition, the controller 130 provides a control signal Ctrl2 to the bias circuit 126 in each of the measurement periods TP1 to TPn, to provide a fixed bias voltage Vb to the sensing unit 125, so that the sensing unit 125 has a fixed magnification.

In response to the control signal Ctrl1, the driver 114 provides a driving current Id with a current value I1 and a drive time TX1 in the measurement period TP1, to generate emitted light EL to illuminate the user 10. Next, the sensing unit 125 receives, from the user 10, reflected light RL corresponding to the emitted light EL in the time interval Td1 after the emitted light EL is generated, where a reception time of the reflected light RL is RX1. In the measurement period TP2, the driver 114 provides a driving current Id with a current value I2 and a drive time TX2, to generate emitted light EL to illuminate the user 10. Then, in the time interval Td2 after the emitted light EL is generated, the sensing unit 125 receives, from the user 10, reflected light RL with a reception time RX2. In the measurement period TP3, the driver 114 provides a driving current Id with a current value I3 and a drive time TX3, to generate emitted light EL to illuminate the user 10. Then, in the time interval Td3 after the emitted light EL is generated, the sensing unit 125 receives, from the user 10, reflected light RL with a reception time RX3. The rest can be deduced by analogy, until the measurement period TPn is reached.

In the embodiment of FIG. 4, the current value I3 is greater than the current value I2, and the current value I2 is greater than the current value I1. In some embodiments, the drive times TX1 to TXn are the same. In some embodiments, the drive times TX1 to TXn may be dynamically adjusted according to a result of analyzing the sensing signal Sn by the controller 130. As previously described, when the driving current Id of the light-emitting unit 112 changes, the wavelength of the emitted light EL drifts. In some embodiments, As the wavelengths are different, the time intervals Td1 to Tdn are different, for example, Td3>Td2>Td1. In addition, the reception times RX1 to RXn of the reflected light RL are also different.

In each of the measurement periods TP1 to TPn, the controller 130 may analyze the received reflected light RL, and obtain spectral distribution curves of corresponding absorption parameters μₐ and scattering parameters μₛ. For example, in the measurement period TP1, the controller 130 may analyze the reflected light RL corresponding to the current value I1 and the drive time TX1 to obtain an optical parameter analysis result (for example, an optical parameter analysis result R1 in FIG. 3) corresponding to the absorption parameter μₐ and the scattering parameter μₛ. Similarly, in the measurement period TP2, the controller 130 may analyze the reflected light RL corresponding to the current value I2 and the drive time TX2 to obtain an optical parameter analysis result (for example, an optical parameter analysis result R2 in FIG. 3) corresponding to the absorption parameter μₐ and the scattering parameter μₛ. In the measurement period TP3, the controller 130 may analyze the reflected light RL of the emitted light EL corresponding to the current value I3 and the drive time TX3 to obtain an optical parameter analysis result (for example, an optical parameter analysis result R3 in FIG. 3) corresponding to the absorption parameter μₐ and the scattering parameter μₛ. The rest can be deduced by analogy. Therefore, the controller 130 may obtain a spectral distribution curve composed of optical parameter analysis results corresponding to a different wavelength (for example, the spectral distribution curve of the absorption parameter μₐ of the glucose concentration C_{g} in FIG. 3), accurately analyze the absorption parameter μₐ and the scattering parameter μₛ, and display information about corresponding physiological signals on the display 160. In some embodiments, the number of the measurement periods TP1 to TPn is determined according to a resolution of a spectral distribution curve (for example, the spectral distribution curve in FIG. 3).

In some embodiments, the controller 130 gradually increases the current value of the driving current Id in each measurement period. For example, the current value I2 is the current value I1 plus a fixed current value ΔI, and the current value I3 is the current value I2 plus the fixed current value ΔI, that is, I2=I1+ΔI and I3=I2+ΔI. In some embodiments, the controller 130 dynamically adjusts a control signal Ctrl1 of a next measurement period according to the reflected light RL that has been received and analyzed so far. For example, according to the reflected light RL received and analyzed in the previous measurement period, the controller 130 may provide a control signal Ctrl1 to the driver 114, to generate a driving current Id with a current value In in the measurement period TPn to drive the light-emitting unit 112, where the current value In is less than the current value I2 and greater than the current value I1. In other words, the controller 130 may dynamically adjust the drive setting (or condition) of the light-emitting unit 112 in each measurement period.

In some embodiments, the light-emitting unit 112 is a (for example, multi-color) single light-emitting diode with a variable wavelength. The controller 130 may provide a control signal Ctrl1 to the light-emitting module 110, to control the light-emitting unit 112 to generate emitted light EL of a different wavelength in a different measurement period. For example, in the measurement period TP1, the controller 130 may control the light-emitting unit 112 to generate emitted light EL with a red light wavelength (for example, a wavelength of 620 nm to 750 nm). In the measurement period TP2, the controller 130 may control the light-emitting unit 112 to generate emitted light EL with a green light wavelength (for example, a wavelength of 495 nm to 570 nm). In the measurement period TP3, the controller 130 may control the light-emitting unit 112 to generate emitted light EL with a blue light wavelength (for example, a wavelength of 450 nm to 495 nm). According to a spectral distribution curve of an absorption parameter μₐ and a scattering parameter μₛ corresponding to a different wavelength interval, the controller 130 can accurately analyze the absorption parameter ula and the scattering parameter μₛ, and display information about corresponding physiological signals on the display 160.

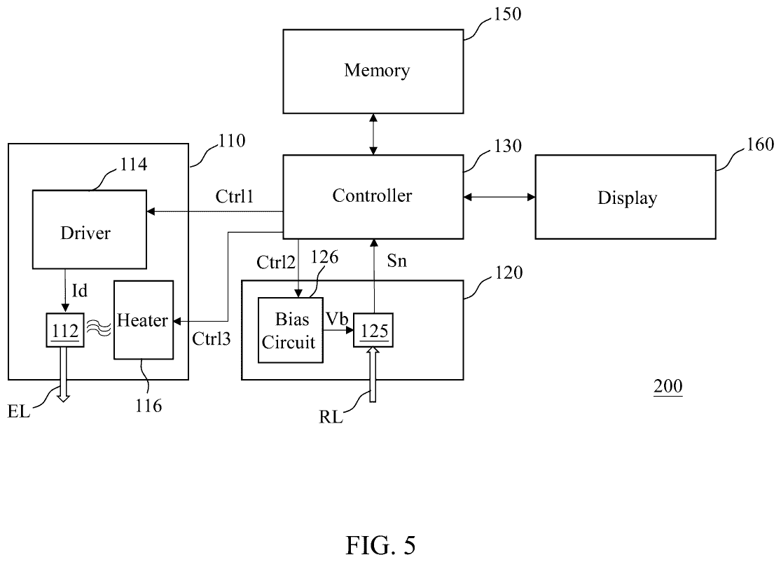

FIG. 5 is a schematic circuit diagram of a physiological signal measuring device 200 according to an embodiment of the present disclosure. A circuit configuration of the physiological signal measuring device 200 in FIG. 5 is similar to that of the physiological signal measuring device 100 in FIG. 2. A difference between the physiological signal measuring device 200 and the physiological signal measuring device 100 is that the light-emitting module 110 in the physiological signal measuring device 200 further includes a heater 116.

In some embodiments, the controller 130 provides a control signal Ctrl1 to the driver 114, to control the driver 114 to provide a fixed driving current Id to the light-emitting unit 112. Meanwhile, the controller 130 provides a control signal Ctrl3 to the heater 116, to heat the light-emitting unit 112. The heater 116 may be a heating device such as a heating pad. When the light-emitting unit 112 has a different temperature, the emitted light EL generated by the light-emitting unit 112 has a different wavelength, that is, the wavelength (for example, a dominant wavelength and a peak wavelength) of the emitted light EL drifts as the temperature changes. For example, when the temperature increases, the wavelength of the emitted light EL drifts toward a long wavelength, for example, the wavelength increases. Conversely, when the temperature decreases, the wavelength of the emitted light EL drifts toward a short wavelength, for example, the wavelength decreases.

In some embodiments, the controller 130 provides a control signal Ctrl1 to the driver 114, to provide different driving currents Id to the light-emitting unit 112. Meanwhile, the controller 130 provides a control signal Ctrl3 to the heater 116, to heat the light-emitting unit 112. Therefore, the controller 130 may change the wavelength of the light-emitting unit 112 by controlling the driving current Id and/or the temperature, to obtain more optical parameter analysis results.

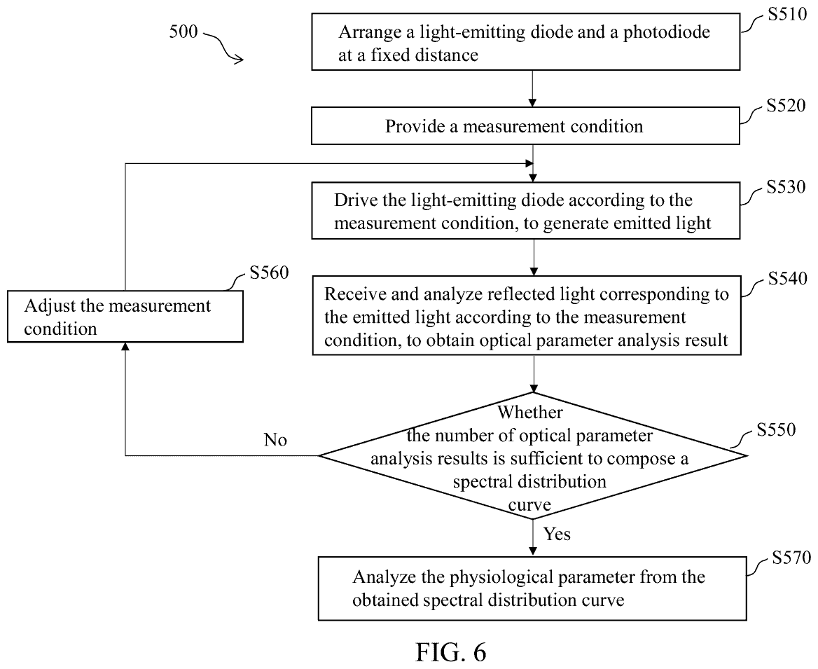

FIG. 6 is a flowchart of a physiological signal measuring method 500 according to an embodiment of the present disclosure. In some embodiments, the physiological signal measuring method 500 of FIG. 6 is performed by the physiological signal measuring device 100 of FIG. 2 or the physiological signal measuring device 200 of FIG. 5.

In operation S510, a light-emitting diode (for example, the light-emitting unit 112) and a photodiode (for example, the sensing unit 125) are arranged at a fixed distance D1 from each other. In some embodiments, the light-emitting diode is a monochrome light-emitting diode. In some embodiments, the light-emitting diode is a multi-color light-emitting diode with a variable wavelength. In some embodiments, the fixed distance D1 is equal to or less than 5 millimeters.

In operation S520, a measurement condition is provided. As previously described, the measurement condition is set by providing the control signals Ctrl1 to Ctrl3 by the controller 130.

In operation S530, the light-emitting diode is driven according to the measurement condition to generate emitted light EL to illuminate a user 10. In some embodiments, the controller 130 may provide the control signal Ctrl1 to control a light-emitting setting of the light-emitting diode, for example, a driving current Id, a drive time, or a light-emitting wavelength of the light-emitting diode, to cause the wavelength of the light-emitting diode to drift. In some embodiments, the controller 130 may provide the control signal Ctrl3 to control a temperature of the light-emitting diode, for example, a junction temperature of the light-emitting diode, to cause the wavelength of the light-emitting diode to drift.

In operation S540, reflected light RL corresponding to the emitted light EL is received according to the measurement condition, and the reflected light RL is analyzed to obtain a corresponding optical parameter analysis result. In some embodiments, the controller 130 may provide the control signal Ctrl2 to control a measurement setting of the photodiode, to cause the photodiode to have a fixed bias voltage, a fixed magnification, and the like.

In operation S550, whether the number of optical parameter analysis results is sufficient to compose a spectral distribution curve of a physiological parameter is determined. For example, whether the number of obtained optical parameter analysis results is equal to or greater than a specific value is determined. In some embodiments, the number of optical parameter analysis results is determined by accuracy of the physiological parameter. In some embodiments, the number of optical parameter analysis results is determined in advance according to a simulation model, and the number of required expressions is stored in the memory 150. For example, through the simulation model, it is determined that at least N optical parameter analysis results of a different wavelength are required to compose a spectral distribution curve. Therefore, the number of expressions may be preset to N and stored in the memory 150.

When the number of optical parameter analysis results is not sufficient to compose a spectral distribution curve (for example, the number of obtained optical parameter analysis results is less than the specific value), the process proceeds to operation S560. In some embodiments, the specific value is 2. In operation S560, the controller 130 adjusts the measurement condition. Then, the process returns to operation S530. In operation S530, the light-emitting diode is re-driven according to the adjusted measurement condition, to generate emitted light EL with a different wavelength to illuminate the user 10. Next, in operation S540, the reflected light RL corresponding to the emitted light EL is received according to the adjusted measurement condition, to obtain a corresponding optical parameter analysis result, until the number of optical parameter analysis results is sufficient to compose a spectral distribution curve in operation S550.

When the number of expressions is sufficient, the process proceeds to operation S570. In operation S570, according to the currently obtained spectral distribution curve, the controller 130 may compare the obtained spectral distribution curve with the known spectral distribution curves (for example, the spectral distribution curves of the absorption parameters μₐ of the different glucose concentrations C_{g} in FIG. 3) in the memory 150, to accurately analyze the physiological parameters, and display physiological signals (for example, blood glucose, blood oxygen, blood pressure, and heart rate) corresponding to the physiological parameters on the display 160 for the user 10 to watch.

The present disclosure provides a physiological signal measuring device and method, to measure a physiological signal of a user through a light-emitting unit 112 and a sensing unit 125 that are arranged at a fixed distance from each other. By controlling the driving current Id of the emitted light EL and/or the temperature in different measurement periods to change the wavelength, spectral distribution curves of an absorption parameter μₐ and a scattering parameter μₛ can be obtained, so that the absorption parameter μₐ and the scattering parameter μₛ can be accurately analyzed according to the spectral distribution curves and a corresponding physiological signal is obtained. In addition, compared with a physiological signal measuring device that needs to use a plurality of light-emitting diodes and a plurality of photodiodes for measurement, the physiological signal measuring device and method provided by the present disclosure only need to use a single light-emitting diode and a single photodiode for measurement of a physiological signal, so that the manufacturing costs and the size can be reduced.

Although the present invention has been described above with reference to the preferred embodiments, the preferred embodiments are not intended to limit the present invention. Any person of ordinary skill in the art may make some variations and modifications without departing from the spirit and scope of the present invention. Therefore, the protection scope of the present invention shall be subject to the appended claims.

What is claimed is:

1. A physiological signal measuring device, comprising:
a light-emitting module, comprising a light-emitting unit configured to generate emitted light according to a first control signal in each measurement period;
a light sensing module, comprising a sensing unit configured to receive reflected light corresponding to the emitted light in each of the measurement periods; and
a controller, configured to change the first control signal in each of the measurement periods to change a wavelength of the emitted light, and obtain an optical parameter analysis result corresponding to the wavelength of the emitted light according to the reflected light,
wherein the controller is configured to obtain a physiological signal corresponding to at least one physiological parameter according to a spectral distribution curve composed of the optical parameter analysis results of the measurement periods.

2. The physiological signal measuring device according to claim 1, wherein the physiological parameter comprises an absorption parameter or a scattering parameter of a user.

3. The physiological signal measuring device according to claim 2, wherein the physiological signal comprises heart rate, blood lipid, bilirubin, lactic acid, respiratory frequency, heart rate variability, core body temperature, body water, alcohol, urea, creatinine, albumin, hemoglobin, glycated hemoglobin, collagen, neutrophil, blood oxygen, blood glucose, or blood pressure of the user.

4. The physiological signal measuring device according to claim 1, wherein the light-emitting unit comprises a single light-emitting diode.

5. The physiological signal measuring device according to claim 4, wherein the controller is configured to provide the first control signal to the light-emitting module in each of the measurement periods to change a driving current of the single light-emitting diode, so as to change a peak wavelength of the emitted light.

6. The physiological signal measuring device according to claim 5, wherein the controller is configured to further provide a third control signal to the light-emitting module in each of the measurement periods to change a temperature of the single light-emitting diode, so as to change the peak wavelength of the emitted light.

7. The physiological signal measuring device according to claim 4, wherein the controller is configured to provide the first control signal to the light-emitting module in each of the measurement periods to change a temperature of the single light-emitting diode, so as to change a peak wavelength of the emitted light.

8. The physiological signal measuring device according to claim 1, wherein the sensing unit comprises a single photodiode.

9. The physiological signal measuring device according to claim 1, wherein a distance between the light-emitting module and the light sensing module is equal to or less than 5 millimeters.

10. The physiological signal measuring device according to claim 1, wherein each of the measurement periods has the same time interval and is less than or equal to one minute.

11. The physiological signal measuring device according to claim 1, wherein a time interval of each of the measurement periods is dynamically adjusted according to the optical parameter analysis results.

12. A physiological signal measuring method, comprising:
arranging a light-emitting diode and a photodiode at a fixed distance;
providing a measurement condition;
driving the light-emitting diode in each measurement period according to the measurement condition, to generate emitted light;
receiving, through the photodiode, reflected light corresponding to the emitted light in each of the measurement periods according to the measurement condition, and obtaining an optical parameter analysis result corresponding to a wavelength of the emitted light according to the reflected light; and
when a quantity of the optical parameter analysis results of the measurement periods is less than a specific value, adjusting the measurement condition in each of the measurement periods, so as to generate emitted light corresponding to the adjusted measurement condition in each of the measurement periods.

13. The physiological signal measuring method according to claim 12, further comprising:
when the quantity of the optical parameter analysis results of the measurement periods is equal to or greater than the specific value, obtaining a physiological signal corresponding to at least one physiological parameter according to a spectral distribution curve composed of the optical parameter analysis results of the measurement periods.

14. The physiological signal measuring method according to claim 13, wherein the physiological signal comprises heart rate, blood lipid, bilirubin, lactic acid, respiratory frequency, heart rate variability, core body temperature, body water, alcohol, urea, creatinine, albumin, hemoglobin, glycated hemoglobin, collagen, neutrophil, blood oxygen, blood glucose, or blood pressure of a user.

15. The physiological signal measuring method according to claim 13, wherein the physiological parameter comprises an absorption parameter or a scattering parameter of a user.

16. The physiological signal measuring method according to claim 12, wherein the driving the light-emitting diode in each measurement period according to the measurement condition, to generate emitted light further comprises:
providing a driving current corresponding to the measurement condition to drive the light-emitting diode to generate the emitted light in each of the measurement periods, wherein a peak wavelength of the emitted light corresponds to the driving current.

17. The physiological signal measuring method according to claim 12, wherein the driving the light-emitting diode in each measurement period according to the measurement condition, to generate emitted light further comprises:
providing a control signal corresponding to the measurement condition to change a temperature of the light-emitting diode to generate the emitted light in each of the measurement periods, wherein a peak wavelength of the emitted light corresponds to the driving current.

18. The physiological signal measuring method according to claim 12, wherein the fixed distance is equal to or less than 5 millimeters.

19. The physiological signal measuring method according to claim 12, wherein each of the measurement periods has the same time interval and is less than or equal to one minute.

20. The physiological signal measuring method according to claim 12, further comprising:
dynamically adjusting a time interval of each of the measurement periods according to the optical parameter analysis results.
